(19) United States
(12) Patent Application Publication
(10) Pub. No.: US 2004/0234979 A1
(43) Pub. Date: Nov. 25, 2004

(54) Differentiall-expressed and up-regulated polynucleotides and polypeptides in breast cancer

(76) Inventors: Zairen Sun, Rockville, MD (US); Gilbert Jay, North Bethesda, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/479,176

(22) PCT Filed: Apr. 1, 2002
(86) PCT No.: PCT/US02/09990

Related U.S. Application Data
(60) Provisional application No. 60/279,678, filed on Mar. 30, 2001.
(60) Provisional application No. 60/293,218, filed on May 25, 2001.

(51) Int. Cl. C12Q 1/68

(52) U.S. Cl. 435/6

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic, etc. The polynucleotides are differentially expressed in cancers, especially breast cancers, and are therefore are useful in variety of ways, including, but not limited to, as molecular markers, as drug targets, and for detecting, diagnosing, staging, monitoring, prognosticating preventing or treating, determining predisposition to, etc., diseases and conditions, such as cancer and other cell-cycle diseases, especially relating to breast. The identification of specific genes, and groups of genes, expressed in a pathway physiologically relevant to cancer permits the definition of disease pathways and the delineation of targets in these pathways which are useful in diagnostic, therapeutic, and clinical applications.

This application claims the benefit of U.S. Provisional Application Ser. No. 60/279,678, filed Mar. 30, 2001, and U.S. Provisional Application Ser. No. 60/293,218, filed May 25, 2001, which are hereby incorporated by reference in their entirety.

### DESCRIPTION OF THE DRAWINGS

FIG. 1 shows the expression patterns of differentially regulated genes in accordance with the present invention. In the top row of each picture, lanes 1-12 represent normal breast epithelium. In the bottom row, lanes 13-24 are from breast cancer tissues. Each lane is an expression pattern from a different patient. The results were obtained according to the following procedures:

Polyadenylated mRNA was isolated individually from breast cancer and normal (control) samples, and used as a template for first-strand cDNA synthesis. The resulting cDNA samples were normalized using beta-actin as a standard. For the normalization procedure, PCR was performed on aliquots of the first-strand cDNA using beta-actin specific primers. The PCR products were visualized on an ethidium bromide stained agarose gel to estimate the quantity of beta-actin cDNA present in each sample. Based on these estimates, each sample was diluted with buffer until each contained the same quantity of beta-actin cDNA per unit volume.

Gene expression was determined in tissues panels comprising both normal breast epithelium and breast cancer. The panels were comprised of 12 normal tissues (upper row, lanes numbered from 1-12) and 12 breast cancer tissues (lower row, lanes numbered from 13-24), each obtained from a different individual. To detect gene expression, PCR was carried out on aliquots of the normalized tissue samples using gene-specific primers (e.g., oligonucleotides comprising from about 22-24 bases). The reaction products were loaded on an agarose (e.g., 1.5-2%) gel and separated electrophoretically. The arrowhead indicates the position of the expected PCR product. The diffuse, but faster migrating, band represents the unused primers. The lane at the far left of each panel contains molecular weight standards.

Table 1 shows a summary of different patterns of differential gene expression observed in breast cancers. Each gene has a sequence identification number (“SEQ NO”), name, expression pattern (“Expr”), Genbank (GI) accession number (“Acc No”), and description. For example, sequence identification number 1 has a GI accession number “532596” and its description indicates it is a human Ig J chain gene. The nucleotide and amino acid sequences for it can be obtained by searching the accession number in GenBank (if only the nucleotide sequence is given, the amino acid sequence can be deduced from it), or, by searching its gene name in GenBank, or any other available database (e.g., Medline, GenSeq, etc.), and recovering the sequences from the database entries (e.g., the GenBank entry or a publication if a literature database, such as Medline, is searched). Although the human name and accession number are listed for sequences in Table 1, other mammalian species are covered, as well, by the entry. For instance, a sequence for a corresponding mouse gene can be obtained, e.g., by searching its gene name in GenBank or any other available database (e.g., Medline, GenSeq, etc.). The nucleotide and amino acid sequences represented in Table 1 are incorporated by reference in their entirety.

SEQ NOS 1-269 indicate genes that are differentially expressed in breast cancer. D=ductal carcinoma in situ (“DCIS”); I=invasive ductal carcinoma (“IDC”); H=high expression; M=medium expression; L=low expression; S=some expression in other tissues not listed. D indicates expression in DCIS, but little or no detectable expression in normal tissue and IDC. I indicates expression in IDC, but little or no detectable expression in normal tissue and DCIS. DI indicates expression in DCIS and IDC, but little or no detectable expression in normal tissue.

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic, etc. The polynucleotides and polypeptides are differentially expressed in cancers, especially breast cancers, and are therefore are useful in variety of ways, including, but not limited to, as molecular markers, as drug targets, and for detecting, diagnosing, staging, monitoring, prognosticating, preventing or treating, determining predisposition to, etc., diseases and conditions, such as cancer and other cell-cycle diseases, especially relating to breast. The identification of specific genes, and groups of genes, expressed in a pathway physiologically relevant to cancer permits the definition of disease pathways and the delineation of targets in these pathways which are useful in diagnostic, therapeutic, and clinical applications.

Breast cancer is the second leading cause of cancer death for all women (after lung cancer), and the leading overall cause of death in women between the ages of 40 and 55. In 2000, several hundred thousand new cases of female invasive breast cancer were diagnosed, and about 40,000 women died from the disease. Nearly 43,000 cases of female in situ (preinvasive) breast cancer were diagnosed in 2000.

There is not one single disease that can be called breast cancer. Instead, it is highly heterogeneous, exhibiting a wide range of different phenotypes and genotypes. No single gene or protein has been identified which is responsible for the etiology of all breast cancers. It is likely that diagnostic and prognostic markers for breast cancer disease will involve the identification and use of many different genes and gene products to reflect its multifactorial origin.

A continuing goal is to characterize the gene expression patterns of the various breast carcinomas in order to genetically differentiate them, providing important guidance in preventing and treating cancer. For instance, the c-erb-B2 gene codes for a transmembrane protein which is over-expressed in about 20-30% of all breast cancers. Based on this information, immunotherapy using an anti-c-erb-B2 antibody has been developed and successfully used to treat breast cancer. See, e.g., Pegram and Slamon, Semin Oncol., 5, Suppl 9:13, 2000. Molecular pictures of cancer, such as the pattern of up-regulated genes identified herein, provide an important tool for molecularly dissecting and classifying cancer, identifying drug targets, providing prognosis and therapeutic information, etc. For instance, an array of polynucleotides corresponding to genes differentially regulated in breast cancer can be used to screen tissue samples for the existence of cancer, to categorize the cancer (e.g., by the particular pattern observed), to grade the cancer (e.g., by the number of up-regulated genes and their amounts of expression), to identify the source of a secondary tumor, to screen for metastatic cells, etc. These arrays can be used in combination with other markers, e.g., keratin immunophenotyping (e.g., CK 5/6), c-erb-B2, estrogen receptor (ER) status, etc., and any of the grading systems used in clinical medicine.

Nucleic Acids

The present invention relates to polynucleotides, such as DNAs, RNAs, and fragments thereof, which are differentially expressed in cancer, especially breast cancer, as compared to normal breast. SEQ NOS 1-269 show the nucleotide sequences of polynucleotides differentially expressed in accordance with the present invention. Table 1 summarizes different patterns of expression. Genes labeled by D, DL, DM, or DH indicate genes whose expression is highly restricted to BCIS or other low grade cancers. Genes labeled by I, IL, IM, or IH indicate genes who expression is highly restricted to IDC or other high grade cancers. Genes labeled by DI, DIL, DIM, or DIH indicate genes who expression is highly restricted to DCIS and IDC, or other low and high grade cancers. These results are for the particular cancers analyzed, DCIS and IDC. Different cancers, including DCIS and IDC obtained from different sources, may have different results. For instance, genes which are described as being up-regulated herein, may be down-regulated, or may even show normal expression levels when examined in other cancers.

By the phrase “differential expression,” it is meant that the levels of expression of a gene, as measured by its transcription or translation product, are different depending upon the specific cell-type. A gene differentially-expressed in DCIS has different expression levels when compared to its expression in IDC and normal tissue. There are no absolute amounts by which the gene expression levels must vary, as long as the differences are measurable.

The phrase “up-regulated” indicates that an mRNA transcript or other nucleic acid corresponding to a polynucleotide of the present invention is expressed in larger amounts in a cancer as compared to the same transcript expressed in normal cells from which the cancer was derived. For instance, a gene's up-regulation can be determined by comparing its abundance per gram of RNA (e.g., total RNA, polyadenylated mRNA, etc.) extracted from a cancer tissue in comparison to the corresponding normal tissue. The normal tissue can be from the same or different individual or source. For convenience, it can be supplied as a separate component or in a kit in combination with probes and other reagents for detecting genes. The quantity by which a nucleic acid is up-regulated can be any value, e.g., more than 10%, 50%, 2-fold, 5-fold, 10-fold, etc. Up-regulation also includes going from substantially no expression, to detectable expression, to significant or highly restricted expression, etc.

The amount of transcript can also be compared to a different gene in the same sample, especially a gene whose abundance is known and substantially no different in its expression between normal and cancer cells (e.g., a “control” gene). If represented as a ratio, with the quantity of up-regulated gene transcript in the numerator and the control gene transcript in the denominator, the ratio would be larger, e.g., in breast cancer than in a sample from normal breast tissue. In general, up-regulation can be assessed by any suitable method, including any of the nucleic acid detection and hybridization methods mentioned below.

Up-regulation can be arise through a number of different mechanisms. The present invention is not bound by any specific way through which it occurs. Up-regulation of a polynucleotide can occur, e.g., by modulating (1) transcriptional rate of the gene (e.g., increasing its rate, inducing or stimulating its transcription from a basal, low-level rate, etc.), (2) the post-transcriptional processing of RNA transcripts, (3) the transport of RNA from the nucleus into the cytoplasm, (4) the RNA nuclear and cytoplasmic turnover (e.g., by virtue of having higher stability or resistance to degradation), and combinations thereof. See, e.g., Tollervey and Caceras, Cell, 103:703-709, 2000.

An up-regulated polynucleotide and polypeptide encoded thereby are useful in a variety of different applications as described in greater details below. Because it is more abundant in cancer, it (or the polypeptide encoded by it, or specific binding partners thereto) can be used as a diagnostic to test for the presence of cancer, e.g., in tissue sections, in a biopsy sample, in total RNA, in lymph or blood, etc. Up-regulated polynucleotides and polypeptides can be used individually, or in groups, to assess the cancer, e.g., to determine the specific type of cancer, its stage of development, the nature of the genetic defect, etc., or to assess the efficacy of a treatment modality. How to use polynucleotides and polypeptides in diagnostic and prognostic assays is discussed below.

In addition, the polynucleotides and the polypeptides they encode, can serve as a target for therapy or drug discovery. A polypeptide, coded for by an up-regulated polynucleotide, which is displayed on the cell-surface, can be a target for immunotherapy to destroy, inhibit, etc., the diseased tissue. Up-regulated transcripts can also be used in drug discovery schemes to identify pharmacological agents which suppress, inhibit, etc., their up-regulation, thereby preventing the phenotype associated with their expression. Thus, an up-regulated polynucleotide of the present invention has significant applications in diagnostic, therapeutic, prognostic, drug development, and related areas.

The expression patterns of the differentially expressed genes disclosed herein can be described as a “fingerprint” in that they are a distinctive pattern displayed by a cancer. Just as with a fingerprint, an expression pattern can be used as a unique identifier to characterize the status of a tissue sample. The list of genes represented by SEQ NOS 1-269 provides an example of a cell expression profile for a breast cancer. It can be used as a point of reference to compare and characterize unknown samples and samples for which further information is sought. Tissue fingerprints can be used in many ways, e.g., to classify an unknown tissue as being a breast cancer, to determine the origin of a particular cancer (e.g., the origin of metastatic cells), to determine the presence of a cancer in a biopsy sample, to assess the efficacy of a cancer therapy in a human patient or a non-human animal model, to detect circulating cancer cells in blood or a lymph node biopsy, etc.

While the expression profile of the complete gene set represented by SEQ NOS 1-269 may be most informative, a fingerprint containing expression information from less than the full collection can be useful, as well. For instance, useful subsets of the genes listed in Table 1, include, but are not limited to, subsets containing only D, I, or DI gene, functional groups, e.g., transcription factors, cell-cycle regulatory proteins, proteases, adhesion proteins, cytokines and cytokine receptors, cell-surface proteins, membrane channels and transporters, enzymes, etc., genes shown in Table 2, etc. In the same way that an incomplete fingerprint may contain enough of the pattern of whorls, arches, loops, and ridges, to identify the individual, a cell expression fingerprint containing less than the full complement may be adequate to provide useful and unique identifying and other information about the sample, e.g., a functional fingerprint of genes have a specific function, such as cytokine or cytokine receptor, cell-cycle associated proteins, etc. Cancer is a multifactorial disease, involving genetic aberrations in more than gene locus. This multifaceted nature may be reflected in different cell expression profiles associated with breast cancers arising in different individuals, in different locations in the same individual, or even within the same cancer focus. As a result, a complete match with a particular cell expression profile, as shown herein, is not necessary to classify a cancer as being of the same type or stage. Similarity to one cell expression profile, e.g., as compared to another, can be adequate to classify cancer types, grades, and stages. Correspondingly, the present invention relates to one or more polynucleotides which are differentially regulated in a breast cancer, selected from: group D (up-regulated in DCIS) genes, SEQ NOS 1-3 and 188-225, for DCIS or a low grade cancer; group I (up-regulated in IDC) genes, SEQ 226-269, for IDC or a high grade cancer; and group DI (up-regulated in DCIS and IDC) genes, SEQ NOS 4-187, for an ungraded cancer.

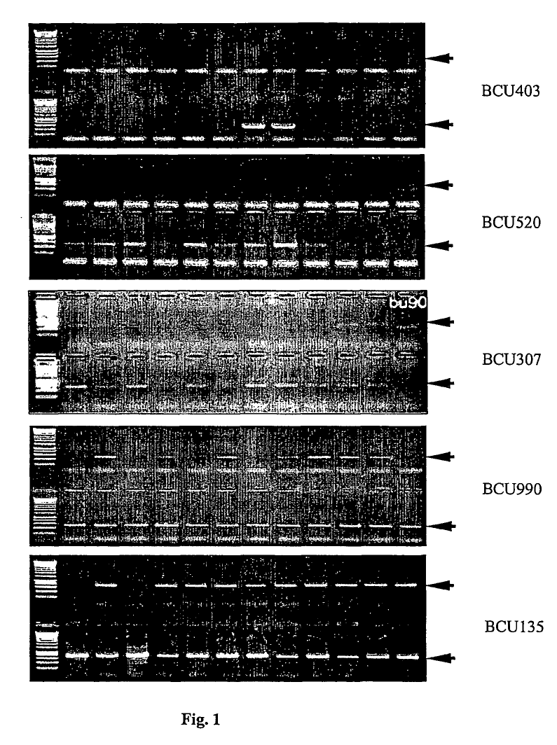

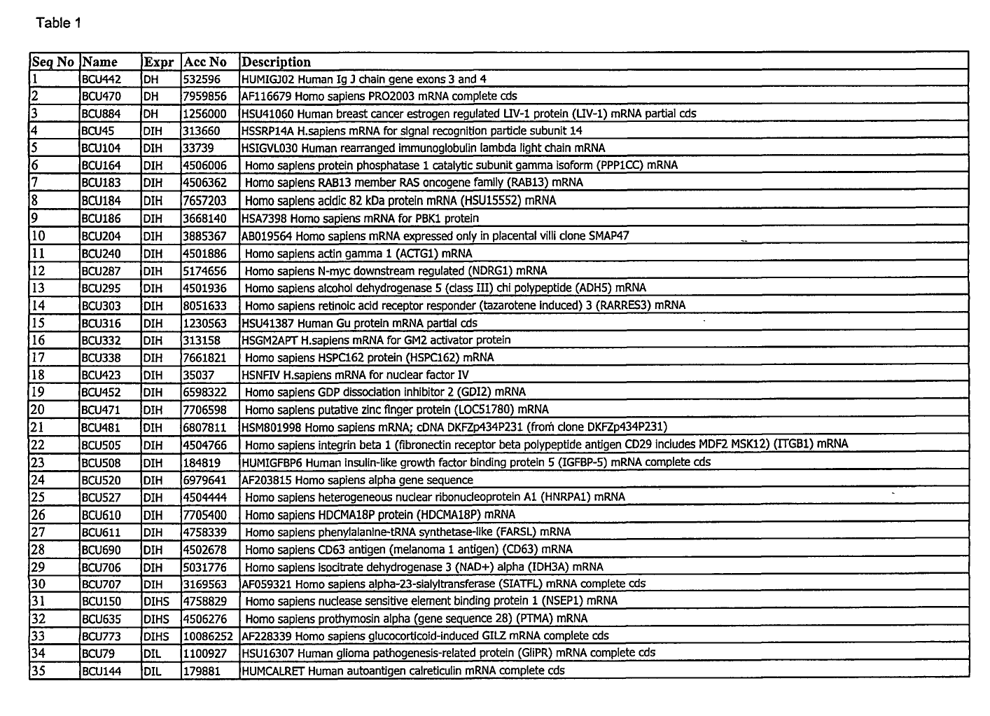

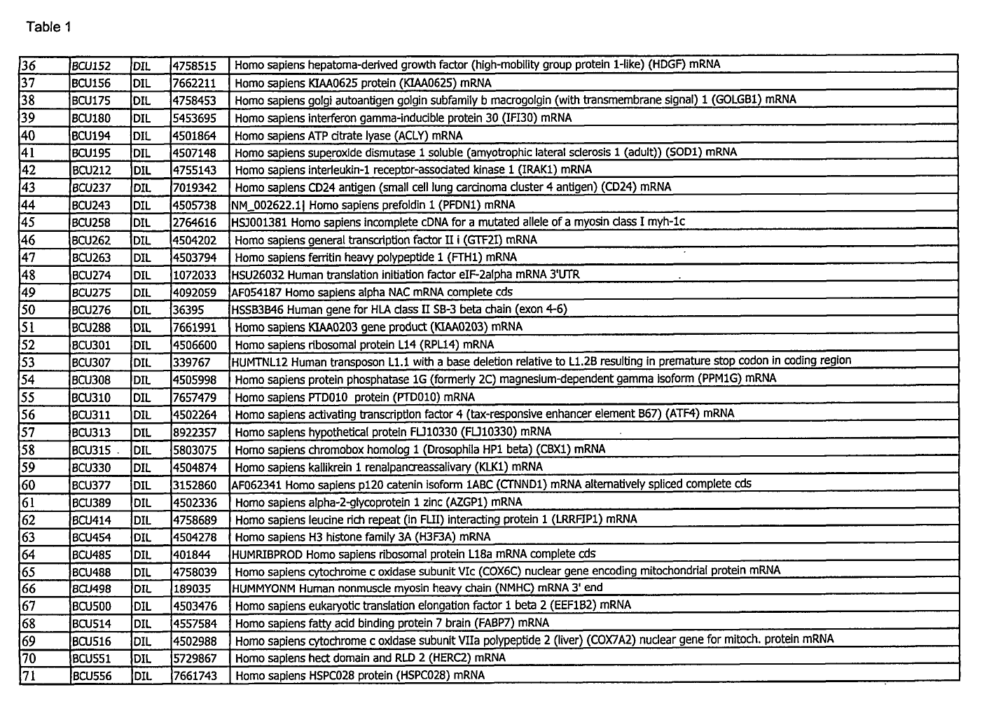

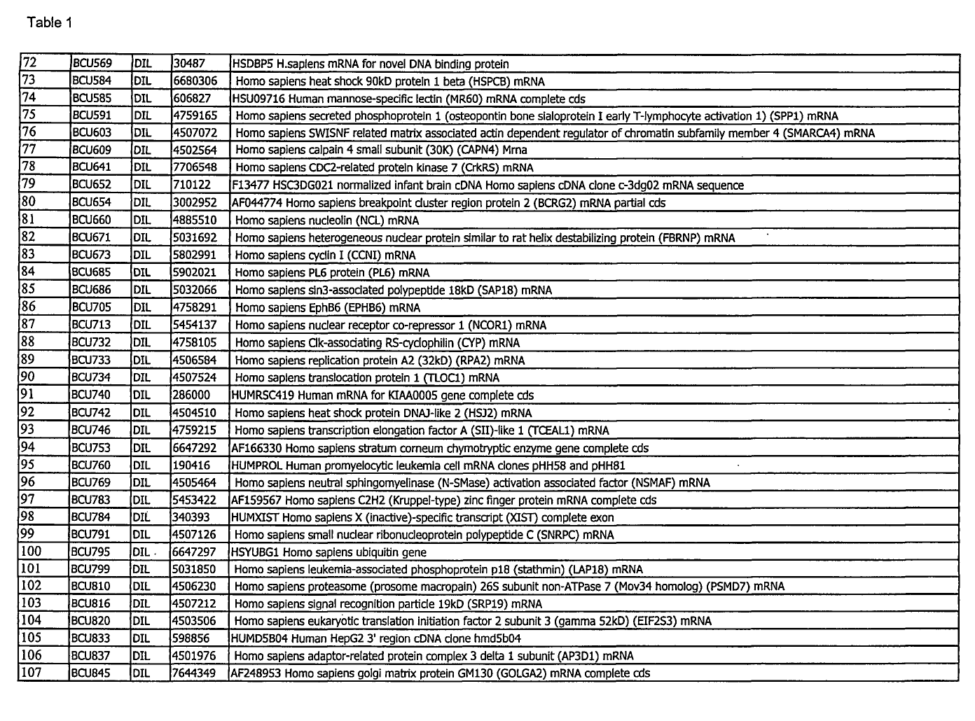

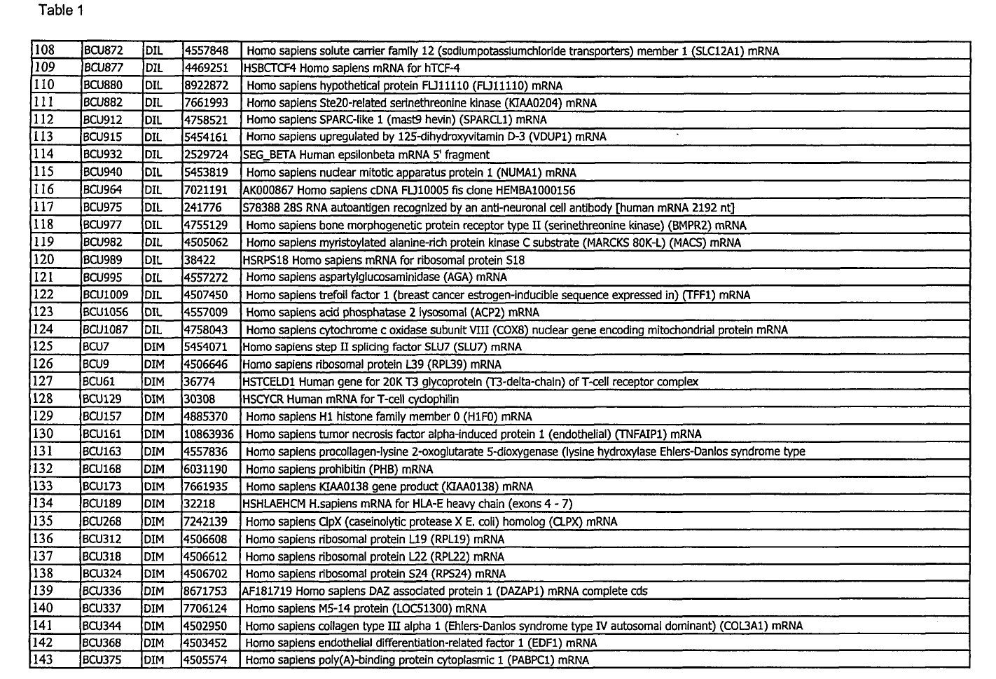

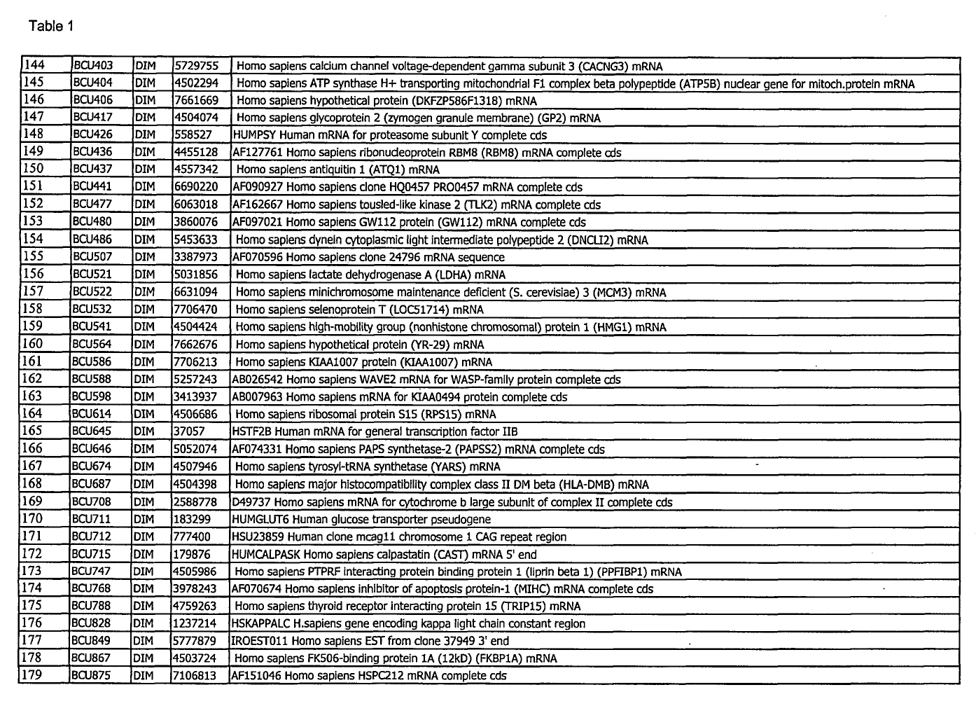

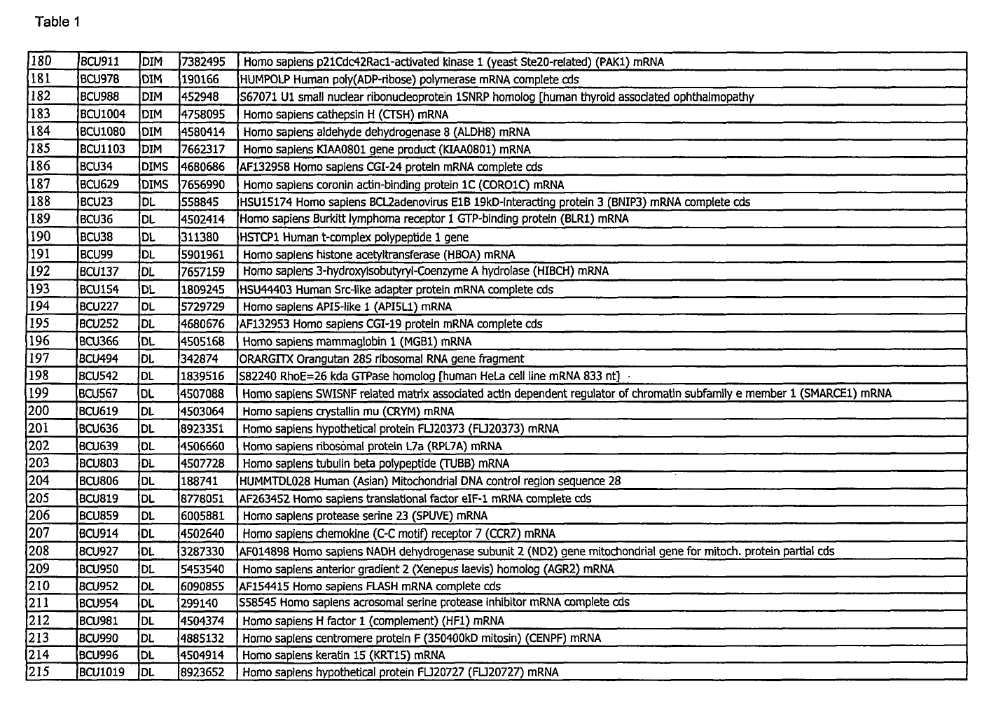

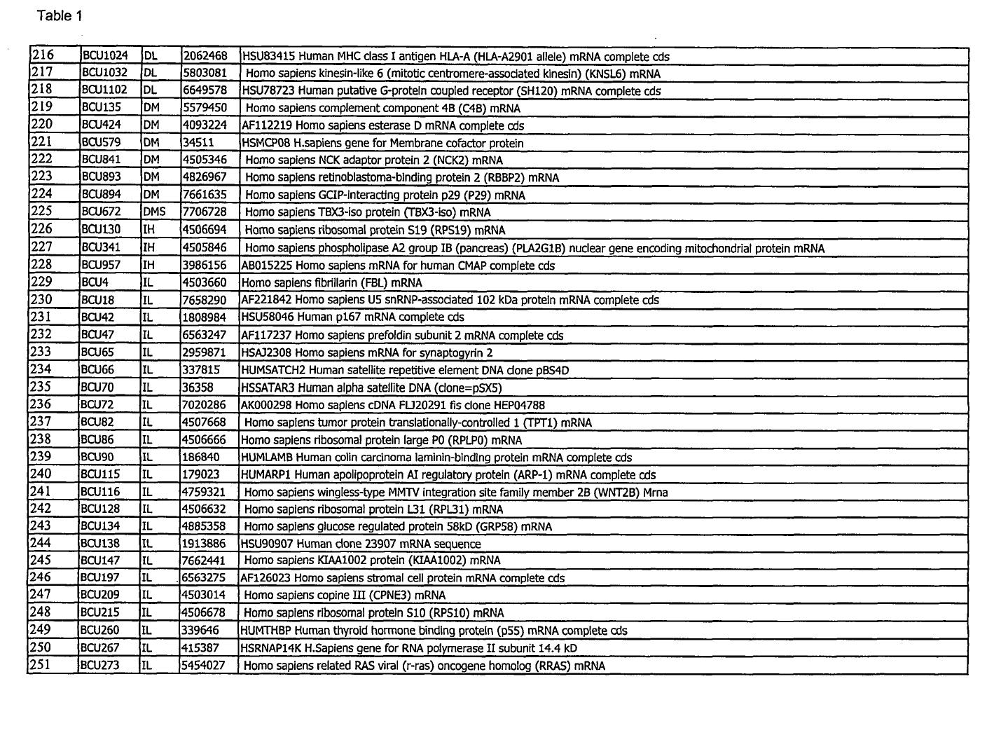

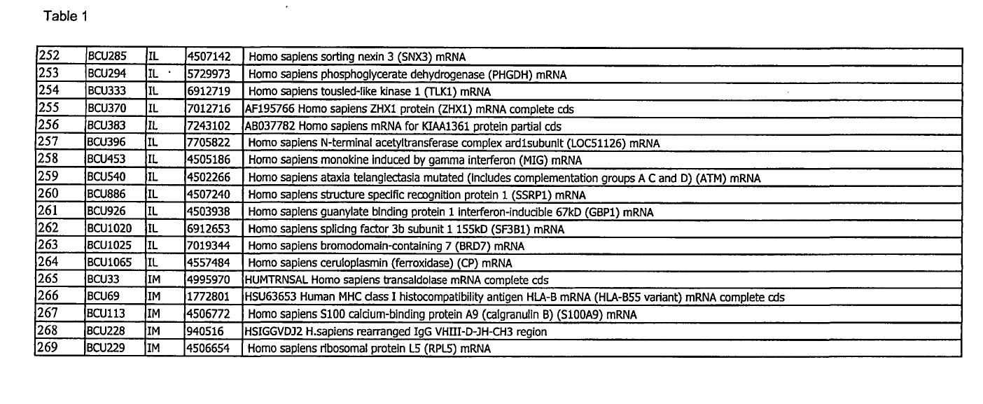

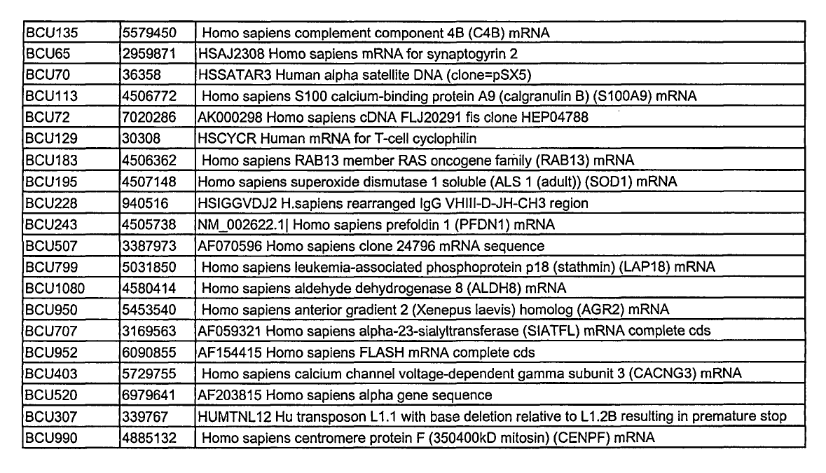

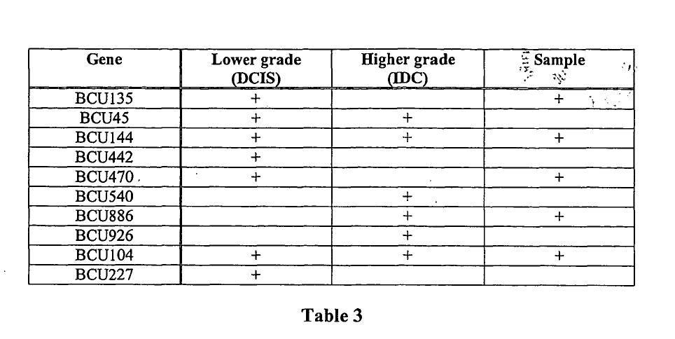

As an illustration, differentially-regulated genes identified herein can be sorted into groups based on their expression patterns. FIG. 1 shows several different possible classes of genes when divided up on the basis of their expression in normal and breast tissues. All these genes are up-regulated in breast cancer. These groups do not limit the utility or application of a gene, but simply provide more information about it.

Class 1 represent genes which do not show significant expression in normal breast epithelium, but which are expressed in one or more breast cancers. Examples include, e.g., BCU403 and BCU520. BCU403 is highly up-regulated in two (lanes 19 and 20) of the 12 breast examined, but shows no significant expression in any of the breast cancers (lanes 1-12). At least 9 of the 12 breast cancers examined exhibited expression of BCU520, while none of the normal breast epithelium did. These genes can be used alone, or together, as diagnostic, therapeutic, or prognostic tools. For instance, since neither gene detects all cancers examined, the polynucleotides (or the products they encode) can be used in combination to increase the number of patents detected or targeted by the genes.

Class 2 genes show significant expression in normal breast epithelium, but up-regulation in breast cancers. There are wide range of expression patterns observed in this class, depending upon their penetrance in normal and cancerous tissues. Gene expression patterns can vary in terms of the number of patients who are marked by the gene, as well as the levels by which such genes are up-regulated. BCU 307 and 990, for instance, show only about 50% expression in normal breast tissue, but over 75% expression in the breast cancers examined. BCU65 and BCU135 show about 100% expression in the breast cancers examined, as well as high penetrance in normal tissues.

A mammalian polynucleotide, or fragment thereof, of the present invention is a polynucleotide having a nucleotide sequence obtainable from a natural source. It therefore includes naturally-occurring normal, naturally-occurring mutant, and naturally-occurring polymorphic alleles (e.g., SNPs), differentially-spliced transcripts, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, or from a cDNA library. The polynucleotide can be obtained from a cell or tissue (e.g., from an embryonic or adult tissues) at a particular stage of development, having a desired genotype, phenotype, disease status, etc.

Polynucleotides can be excluded from methods, processes, etc., of the present invention if, e.g., such methods were known on the day this application was filed and/or disclosed in a patent application having an earlier filing or priority date than this application and/or conceived and/or reduced to practice earlier than a polynucleotide in this application. The entire set of polynucleotides disclosed herein can be claimed, and subsets thereof, including any combination or permutation thereof, such as subsets containing only 1 member.

As explained in more detail below, a polynucleotide sequence of the invention can contain the complete sequence as represented by SEQ NOS 1-269, degenerate sequences thereof, anti-sense, muteins thereof, genes comprising said sequences, full-length cDNAs comprising said sequences, fragments thereof, homologs, primers, derivatives thereof, nucleic acid molecules which hybridize thereto, genomic DNA, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions can be identified upstream of coding and expressed RNAs, and assayed routinely for activity, e.g., by joining to a reporter gene (e.g., CAT, GFP, alkaline phosphatase, luciferase, galatosidase). A promoter obtained from a breast-selective gene can be used, e.g., in gene therapy to obtain tissue-specific expression of a heterologous gene (e.g., coding for a therapeutic product or cytotoxin). Because of efforts in the sequencing of the entire human genome, many genomic sequences were known at the time of filing this application and are incorporated by reference in their entirety, e.g., Nature, 409, 860-921 (2001), Science, Volume 291, No. 5507 (Feb. 16, 2001).

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR540, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Nucleic acid hybridization technology is useful for variety of different purposes and formats, e.g., to select homologs of genes listed in Table 1, to screen for gene expression profile, to ascertain information about gene expression, to diagnose, to obtain genomic clones, etc.

A polynucleotide in accordance with the present invention can be selected on the basis of polynucleotide hybridization. The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ NOS 1-269 and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in SEQ NOS 1-269 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ NOS 1-269 or genomic sequences thereof, under low or high stringency conditions.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5 +16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complementarity between the probe (e.g., a short polynucleotide of SEQ NOS 1-269 or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloninig, Chapter 11, 1989. Such homologs can have varying amounts of nucleotide and amino acid sequence identity and similarity to such polynucleotides of the present invention. Mammalian organisms include, e.g., mouse, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Hybridization, as discussed above and below, is useful in a variety of applications, including, in gene detection methods, for identifying mutations, for making mutations, to identify homologs in the same and different species, to identify related members of the same gene family, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various coimnercial and free sources of alignment programs are available, e.g., MegAlign by DNA Star, BLAST (National Center for Biotechnology Information), etc.

Percent sequence identity can also be determined by conventional methods, e.g., as described in Altschul et al., Bull. Math. Bio. 48: 603-616, 1986 and Henikoff and Henikoff, Proc. Natl. Acad. Sci. USA 89:10915-10919, 1992.

Polypeptides

A mammalian polypeptide of the present invention is a full-length mammalian polypeptide having an amino acid sequence which is obtainable from a natural source. Polypeptides include those coded for by the genes listed in Table 1, and include all mammalian homologs, such as human, mouse, and rat. Also included are naturally-occurring normal, naturally-occurring mutant, and naturally-occurring polymorphisms, including single nucleotide polymorphisms (SNP), differentially-spliced transcripts, etc., sequences. Natural sources include, e.g., living cells, e.g., obtained from tissues or whole organisms, cultured cell lines, including primary and immortalized cell lines, biopsied tissues, etc.

The present invention also relates to fragments of a mammalian polypeptide. The fragments are preferably “biologically active.” By “biologically active,” it is meant that the polypeptide fragment possesses an activity in a living system or with components of a living system. Biological activities include, e.g., protein-specific immunogenic activity. A “protein-specific immunogenic activity” means, e.g., that a polypeptide derived from the protein elicits an immunological response that is selective for the protein. This immunological response can include one or more cellular and/or humoral components, e.g., the stimulation of antibodies, T-cells, macrophages, B-cells, dendritic cells, etc. Immunological responses can be measured routinely.

Fragments can be prepared according to any desired method, including, chemical synthesis, genetic engineering, cleavage products, etc. A biologically-fragment includes, e.g., polypeptide which have had amino acid sequences removed or modified at either the carboxy- or amino-terminus of the protein.

Polypeptides of the present invention can be analyzed by any suitable methods to identify other structural and/or functional domains in the polypeptide, including membrane spanning regions, hydrophobic regions. For example, a mammalian polypeptide can be analyzed by methods disclosed in, e.g., Kyte and Doolittle, J. Mol. Bio., 157:105, 1982; EMBL Protein Predict; Rost and Sander, Proteins, 19:55-72, 1994.

Other homologs of polypeptides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polypeptide. Mammalian organisms include, e.g., human, mouse, rats, monkeys, pigs, sheep, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc.

A polypeptide of the present invention can also have 100% or less amino acid sequence identity to an amino acid sequence coded for by a mammalian gene set forth in Table 1. For the purposes of the following discussion: Sequence identity means that the same nucleotide or amino acid which is found in a target sequence is found at the corresponding position of the compared sequence(s). A polypeptide having less than 100% sequence identity to the amino acid sequences can contain various substitutions from the naturally-occurring sequence, including homologous and non-homologous amino acid substitutions. See below for examples of homologous amino acid substitution. The sum of the identical and homologous residues divided by the total number of residues in the sequence over which the polypeptide is compared is equal to the percent sequence similarity. For purposes of calculating sequence identity and similarity, the compared sequences can be aligned and calculated according to any desired method, algorithm, computer program, etc., including, e.g., BLAST. A polypeptide having less than 100% amino acid sequence identity to polypeptide coded for by a gene set forth in Table 1 can have about 99%, 98%, 97%, 95%, 90%, 90%, 87% 85%, 80%, 75%, 70%, 65%, 60%, 55%, 50%, sequence identity or similarity.

The present invention also relates to polypeptide muteins. By the “mutein,” it is meant any polypeptide which has an amino acid sequence which differs in amino acid sequence from an amino acid sequence obtainable from a natural source (a fragment of a mammalian polypeptide of the present invention does not differ in amino acid sequence from a naturally-occurring polypeptide although it differs in amino acid number). Thus, polypeptide muteins comprise amino acid substitutions, insertions, and deletions, including non-naturally occurring amino acids.

Muteins to a polypeptide sequence of the invention can also be prepared based on homology searching from gene data banks, e.g., Genbank, EMBL. Sequence homology searching can be accomplished using various methods, including algorithms described in the BLAST family of computer programs, the Smith-Waterman algorithm, etc. A mutein(s) can be introduced into a sequence by identifying and aligning amino acids within a domain which are identical and/or homologous between polypeptides and then modifying an amino acid based on such alignment. When a conserved or homologous amino acid is replaced by a non-homologous amino acid, such replacement or substitution can be expected to reduce, decrease, eliminate, or increase a biological activity. For instance, where alignment reveals identical amino acids conserved between two or more domains, elimination or substitution of the amino acid(s) would be expected to affect its biological activity. The effects of such mutations on activity can be determined by various assays described below and as a skilled worker would know.

Amino acid substitution can be made by replacing one homologous amino acid for another. Homologous amino acids can be defined based on the size of the side chain and degree of polarization, including, small nonpolar: cysteine, proline, alanine, threonine; small polar: serine, glycine, aspartate, asparagine; large polar: glutamate, glutamine, lysine, arginine; intermediate polarity: tyrosine, histidine, tryptophan; large nonpolar: phenylalanine, methionine, leucine, isoleucine, valine. Homologous acids can also be grouped as follows: uncharged polar R groups, glycine, serine, threonine, cysteine, tyrosine, asparagine, glutamine; acidic amino acids (negatively charged), aspartic acid and glutamic acid; basic amino acids (positively charged), lysine, arginine, histidine. Homologous amino acids also include those described by Dayhoff in the Atlas of Protein Sequence and Structure 5, 1978, and by Argos in EMBO J., 8, 779-785, 1989.

A mammalian polypeptide of the present invention, fragments, or substituted polypeptides thereof, can also comprise various modifications, where such modifications include lipid modification, methylation, phosphorylation, glycosylation, covalent modifications (e.g., of an R-group of an amino acid), amino acid substitution, amino acid deletion, or amino acid addition. Modifications to the polypeptide can be accomplished according to various methods, including recombinant, synthetic, chemical, etc.

Polypeptides of the present invention (e.g., full-length, fragments thereof, mutations thereof) can be used in various ways, e.g., in assays, as immunogens for antibodies as described below, as biologically-active, as inhibitors, etc.

A polypeptide of the present invention, a derivative thereof, or a fragment thereof, can be combined with one or more structural domains, functional domains, detectable domains, antigenic domains, and/or a desired polypeptide of interest, in an arrangement which does not occur in nature, i.e., not naturally-occurring. A polypeptide comprising such features is a chimeric or fusion polypeptide. Such a chimeric polypeptide can be prepared according to various methods, including, chemical, synthetic, quasi-synthetic, and/or recombinant methods. A chimeric polynucleotide coding for a chimeric polypeptide can contain the various domains or desired polypeptides in a continuous (e.g., with multiple N-terminal domains to stabilize or enhance activity) or interrupted open reading frame, e.g., containing introns, splice sites, enhancers, etc. The chimeric polynucleotide can be produced according to various methods. See, e.g., U.S. Pat. No. 5,439,819. A domain or desired polypeptide can possess any desired property, including, a biological function such as signaling, growth promoting, cellular targeting (e.g., signal sequence, targeting sequence, such as targeting to the endoplasmic reticulum or nucleus), etc., a structural function such as hydrophobic, hydrophilic, membrane-spanning, etc., receptor-ligand functions, and/or detectable functions, e.g., combined with enzyme, fluorescent polypeptide, green fluorescent protein, (Chalfie et al., Science, 263:802, 1994; Cheng et al., Nature Biotechnology, 14:606, 1996; Levy et al., Nature Biotechinology, 14:610, 1996), etc. In addition, a polypeptide, or a part of it, can be used as a selectable marker when introduced into a host cell. For example, a polynucleotide coding for an amino acid sequence according to the present invention can be fused in-frame to a desired coding sequence and act as a tag for purification, selection, or marking purposes. The region of fusion can encode a cleavage site to facilitate expression, isolation, purification, etc.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6×His, maltose binding protein, chitinase, etc) and then purify by anti-tag antibody-conjugated affinity chromatography.

Many methods of using polypeptides may require that it be labeled. Any modification or label which is effective to achieve detection can be used, including, e.g., avidin, biotin, radioactive atoms, fluorescent tags, enzyme tags, polypeptide tags, chemiluminescent, electrochemiluminescent, biotin, and fret pairs among others.

Nucleic Acid Detection Methods

Another aspect of the present invention relates to methods and processes for detecting and assessing cancer in a sample using a polynucleotide in accordance with the present invention. Such a polynucleotide can also be referred to as a “probe.” The term “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. No. 712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any methods suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection. Including, e.g., avidin, biotin, radioactive atoms, fluorescent tags, enzyme tags, polypeptide tags, etc.

Detection can be desirable for a variety of different purposes, including research, diagnostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, etc.

Detection can be accomplished in combination with polynucleotide probes for other genes, e.g., genes which are differentially expressed in other tissues and cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, breast, fat, adult and embryonic stem cells, specific cell-types, such as neurons, fibroblasts, myocytes, mesenchymal cells, etc.

Specific Probes

A polynucleotide probe of the present invention can comprise any continuous nucleotide sequence of SEQ NOS 1-269, sequences which share sequence identity thereto, or complements thereof. These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-100, 8-700, 8-600, 8-500, 8-400, 8-300, 8-150, 8-100, 8-75 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of SEQ NOS 1-269, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for genes differentially expressed in cancer, e.g., comprising a forward and reverse primer effective in PCR. These include both sense and anti-sense orientations. For instance, in PCR-based methods, a pair of primers are typically used, one having a sense sequence and the other having an antisense sequence.

Another aspect of the present invention is a nucleotide sequence that is specific to, or for, a selective polynucleotide. The phrase “specific sequence” to, or for, a polynucleotide, has a functional meaning that the polynucleotide can be used to identify the presence of one or more target genes in a sample. It is specific in the sense that it can be used to detect polynucleotides above background noise (“non-specific binding”). A specific sequence is a defined order of nucleotides which occurs in the polynucleotide, e.g., in the nucleotide sequences of SEQ NOS 1-269. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 95% identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of breast in a blood sample is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, and mixtures thereof A polynucleotide can be single-, or double-stranded, triplex, e.g., dsDNA, DNA:RNA, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, 8-oxo-guanine.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985), linker-scanning (McKnight and Kingsbury, Science, 217:316-324, 1982), directed using PCR, etc. Desired sequences can also be produced by the assembly of target sequences using mutually priming oligonucleotides (Uhlmann, Gene, 71:29-40, 1988).

Methods of Using Probes, Polynucleotides, etc

Probes, polynucleotides, antibodies, and specific binding partners can be used in wide range of methods and compositions, including for detecting, diagnosing, staging, grading, assessing, etc., cancer, for monitoring or assessing therapeutic and/or preventative measures, in ordered arrays, etc.

Along these lines, the present invention relates to methods of detecting breast cancer cells in a sample comprising nucleic acid, comprising one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is SEQ NOS 1-269, a polynucleotide having, e.g., about 70%, 80%, 85%, 90%, 95%, 99%, or more sequence identity thereto, or effective fragments thereof, and said polynucleotide is differentially expressed in said breast. The detection method includes, e.g., detecting the presence of cancer cells in a sample, and diagnosing cancer, e.g., in a tissue biopsy, blood, urine, stool, and other bodily fluids and samples.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used herein, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in SEQ NOS 1-269 is desired, a probe can be selected which can hybridize to such target gene under high stringent conditions, without significant hybridization to other genes in the sample. To detect homologs of a polynucleotide set forth in SEQ NOS 1-269, the effective hybridization conditions can be less stringent, and/or the probe can comprise codon degeneracy, such that a homolog is detected in the sample.

As already mentioned, the method can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining whether polynucleotides of the present invention are differentially expressed in a cancer as compared to the same gene in a normal tissue. Such methods can involve substantially the same steps as described above for presence/absence detection, e.g., contacting with probe, hybridizing, and detecting hybridized probe. Rather than simply assessing whether probe is bound to its target, these methods can further comprise, e.g., detecting the amount of hybridization between said probe and target nucleic acid; determining by said hybridization whether said target nucleic acid is up-regulated in said sample, whereby the presence of an up-regulated target nucleic acid indicates that said sample comprises cancer cells, wherein said probe is a polynucleotide which is SEQ NOS 1-269, a polynucleotide having 95% sequence identity or more to a sequence set forth in SEQ NOS 1-269, effective specific fragments thereof, complements thereto.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain cancer cells. Usually, the comparison will be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

The present invention also relates to methods of detecting, diagnosing, staging, grading, determining, etc., a breast cancer in a sample comprising breast cancer, comprising, e.g., determining the number of target genes which are differentially expressed (e.g., up-regulated, down-regulated) in said sample, wherein said target genes comprise a gene which is represented by a sequence selected from SEQ NOS 1-269, or, a gene represented by a sequence having 95% sequence identity or more to a target genes are selected from SEQ NOS 1-269, wherein said genes are up-regulated in breast cancer, and whereby said number is indicative of the probability that, e.g., said sample comprises breast cancer, said sample comprises a cancer at a particular stage, said sample comprises a particular grade of cancer cells (e.g., describing the appearance and behavior of the cells, e.g., as atypical, the derivation of the cells (e.g., carcinoma, sarcoma, etc.), dysplasia, granuloma, hyperplasia, metaplasia, etc.)

A goal, among others, of the method is to determine the presence of breast cancer cells in a sample of any origin, and/or to characterize the nature or origin (i.e., derivation) of cancer cells once identified. This can be accomplished by deciding whether one or more genes in a set of target genes are differentially expressed in the sample of interest. Although the genes are, as a group, differentially expressed in breast cancer, because of variability between individuals and tissue samples, each gene may not be expressed 100% of the time in all breast cancer. There are many sources of variability that account for differences in gene penetrance between individual cancers, including, the developmental and physiological state of the tissue and cells (e.g., hyperplastic, dysplastic, neoplastic, malignant, benign, metastatic, inflamed, etc), cell cycle status, effects of other genes, environmental effects, age, health, gender, existence of other physiological conditions, etc. As a result, it may be advantageous to determine the expression of more than one gene to obtain the maximal amount of information to diagnose the presence of the cancer and its physiological status. In view of the multifactorial nature of cancer, this may be especially advantageous. Methods and compositions of the present invention correspondingly relate to the differentially expressed genes described herein as a group or panel as a reagent to diagnose, stage, grade, etc., a cancer in much the same way that a fingerprint is used as a unique identifier of an individual. Fingerprints can be useful even when a complete print is unavailable. Similarly, an expression profile showing a subset of the differentially expressed polynucleotides can be useful and diagnostic, depending, e.g., on which genes are measured and their contribution to the phenotype. Different stages, grades, etc., of a cancer may have different gene expression fingerprints, but may share subsets of differentially expressed genes represented by SEQ NOS 1-269, e.g., differentially expressing a subset of the genes, differing in the quantity of differential expression detected.

By the term “diagnose” or “diagnosing,” it is meant that it is determined whether a cancer is present in the sample and/or the cancer's grade, stage, or other cancer status indicator. As discussed above, because of individual variability and gene penetrance, certainty or probability that a given sample is a breast cancer can be correlated with the number of differentially expressed genes in the sample. Successive probes can be chosen based on their specificities. A greater number of genes determined to be expressed in a sample can indicate that there is a higher probability that the sample comprises breast cancer. Probability values can be determined statistically and/or empirically, e.g., by making many measurements on individuals in a given population and determining the frequency in which the gene is expressed. These values can differ, depending upon the selected population, e.g., gender, health, ancestry, age, etc.

By the phrase “target genes,” it is meant the genes that the method is aimed at determining. Each of the nucleotide sequences shown in SEQ NOS 1-269 represents a region of a target gene, i.e., a fragment of a complete gene (e.g., a gene has regulatory and coding sequences) serving as a specific identification label for that target gene, and can be referred to as representing a specific gene.

The expression of the genes in a sample can be determined by any effective method. The term “expression” means, e.g., transcription of the gene into RNA, or translation of an RNA into protein. Expression can be determined, e.g., by detecting RNA, by detecting polypeptide translated from the RNA, or any product produced during expression of the gene. Nucleic acid and polypeptide detection are routine, and can be accomplished as described herein or as the skilled worker would know. For example, detecting of RNA can be performed by Northern blot analysis, polymerase chain reaction (PCR), reverse transcriptase PCR, RACE PCR, or in situ hybridization using a polynucleotide probe which is SEQ NOS 1-269, a polynucleotide having sequence identity thereto, effective specific fragments thereof, complements thereto, and said polynucleotide is differentially expressed in said breast. Any amount of sequence identity is suitable as long as it maintains the desired amount of specificity.

Assessing the effects of drugs, radiation therapy, and other therapeutic and prophylactic interventions (e.g., administration of a drug, chemotherapy, etc.) on a cancer is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, to analyzing new animal models, and in any scenario involving cancer treatment and prevention. Analyzing the gene expression profiles of polynucleotides of the present invention can be utilized as a parameter by which interventions are judged and measured. For example, SEQ NOS 1-269 provide a list of sequences that represent genes up-regulated in a breast cancer. Treatment of the cancer, for instance by administration of an anti-neoplastic drug, may change the expression profile in some manner which is prognostic or indicative of the drug's effect on the cancer. Changes in the profile can indicate, e.g., drug toxicity (e.g., by altering the expression of genes not part of the cancer fingerprint), or, a return to a normal, state (e.g., if one or more genes up-regulated in the cancer return to expression levels characteristic of normal tissue, rather than a cancer). Accordingly, the present invention also relates to methods of monitoring or assessing a therapeutic or preventative measure (e.g., chemotherapy, radiation, anti-neoplastic drugs, antibodies, etc.) in a subject having a cancer, or, susceptible to a cancer, comprising, e.g., detecting the expression levels of differentially expressed target genes, where the target genes comprise a gene which is represented by a sequence selected from SEQ NOS 1-269, or, a gene represented by a sequence having 95% sequence identity or more to a sequence selected from SEQ NOS 1-269. A subject can be a cell-based assay system, non-human animal model, human patient, etc. Detecting can be accomplished as described for the methods above and below.

Polynucleotides of the present invention can also be utilized to identify mutant alleles, SNPs, and other polymorphisms of the wild-type gene. Mutant alleles, polymorphisms, SNPs, etc., can be identified and isolated from cancers that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from SEQ NOS 1-269 is used as a probe. The selected mutant alleles, SNPs, polymorphisms, etc., can be used diagnostically to determine whether a subject has, or is susceptible to cancer, as well as to design therapies and predict the outcome of the disease. Methods involve, e.g., diagnosing a cancer, comprising, detecting the presence of a mutation in a gene represented a polynucleotide selected from SEQ NOS 1-269. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

Specific Binding Partners

The present invention also relates to specific-binding partners, such as antibodies, lectins, and aptamers, that specifically recognize a polynucleotide or polypeptide of the present invention. A specific-binding partner is a molecule, which through chemical or physical forces, selectively binds or attaches to a polynucleotide or polypeptide. Specific binding partners generally are referred to in pairs, e.g., antigen and antibody, ligand and receptor. The same general definitions, compositions, and methods which are described for antibodies, applies to other classes of specific-binding partners, as well.

An antibody specific for a polypeptide means that the antibody recognizes a defined sequence of amino acids within or including the polypeptide. Thus, a specific antibody will generally bind with higher affinity to an amino acid sequence of a defined than to a different epitope(s), e.g., as detected and/or measured by an immunoblot assay or other conventional immunoassay. Thus, an antibody which is specific for an epitope of a polypeptide is useful to detect the presence of the epitope in a sample, e.g., a sample of tissue containing human polypeptide product, distinguishing it from samples in which the epitope is absent. Such antibodies are useful as described in Santa Cruz Biotechnology, Inc., Research Product Catalog, and can be formulated accordingly.

Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. For example, for the production of monoclonal antibodies, a human or mouse polypeptide coded for by a gene listed in Table 1 can be administered to mice, goats, rabbits, chickens, etc., subcutaneously and/or intraperitoneally, with or without adjuvant, in an amount effective to elicit an immune response. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, sheep, rat, chicken (e.g., IgY; see, Duan, WO/029444 for methods of making antibodies in avian hosts, and harvesting the antibodies from the eggs).

Polypeptides for use in the induction of antibodies do not need to have biological activity; however, they have immunogenic activity, either alone or in combination with a carrier. Polypeptides used to elicit specific antibodies may have an amino sequence consisting of at least five amino acids, preferably at least 10 amino acids. Short stretches of amino acids, e.g., five amino acids, can be fused with those of another protein such as keyhole limpet hemocyanin, or another useful carrier, and the chimeric molecule used for antibody production. Regions of the polypeptides useful in making antibodies can be selected empirically, or, e.g., an amino acid sequence, as deduced from the cDNA, can be analyzed to determine regions of high immunogenicity. Analysis to select appropriate epitopes is described, e.g., by Ausubel F M et al., Current Protocols in Molecular Biology, Volume 2, 1989, John Wiley & Sons).

The polypeptides and antibodies of the present invention may be used with or without modification. Frequently, the polypeptides and antibodies will be labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; and 4,366,241.

Antibodies and other specific-binding partners which bind polypeptide can be used in various ways, including as therapeutic, diagnostic, and commercial research tools, e.g., to quantitate the levels of polypeptide in animals, tissues, cells, etc., to identify the cellular localization and/or distribution of it, to purify it, or a polypeptide comprising a part of it, to modulate the function of it, in Western blots, ELISA, dot blot, immunoprecipitation, RIA, FACS analysis, etc. The present invention relates to such assays, compositions and kits for performing them, etc. Utilizing these and other methods, an antibody according to the present invention can be used to detect polypeptide or fragments thereof in various samples, including tissue, cells, body fluid, blood, urine, cerebrospinal fluid.

In addition, ligands which bind to a polypeptide according to the present invention, or a derivative thereof, can also be prepared, e.g., using synthetic peptide libraries or aptamers (e.g., Pitrung et al., U.S. Pat. No. 5,143,854; Geysen et al., J. Immunol. Methods, 102:259-274, 1987; Scott et al., Science, 249:386, 1990; Blackwell et al., Science, 250:1104, 1990; Tuerk et al., 1990, Science, 249: 505).

Tissue and Disease

The normal female breast comprises ducts and lobuloalveolar structures surrounded by basement membranes and collagenous stroma with fibroblasts, vessels, and fat. The basic unit of function in the breast are the lobuloalveolar structures which produce the milk secretions. Each lobule drains into a lactiferous duct that empties into a lactiferous sinus beneath the nipple. The ducts are lined with epithelial cells, containing few mitochondria and sparse endoplasmic reticulum. The lobules contain luminal epithelial cells, basal epithelial cells, and myoepithelial cells. The basal and epithelial cells are sometimes grouped together. The luminal cells can be differentiated immuno-histochemically from the myoepithelial cells by their expression of keratins. The luminal cells stain with antibodies to keratin 5/6; the myoepithelial cells stains with antibodies against keratin 8/18. In addition to the presence of these cells types in the breast, there are endothelial cells associated with blood vessels, stromal cells that surround the lobular structures, adipose cells, and blood cells, such as T-lymphocytes and macrophages.

Breast carcinoma can be classified into two basic types, noninvasive (non-infiltrating) and invasive. Noninvasive carcinoma includes, e.g., intraductal carcinoma (also known as ductal carcinoma in situ or “DCIS”), intraductal papillary carcinoma, and lobular carcinoma in situ. Invasive carcinoma includes, e.g., invasive ductal carcinoma (“IDC”), invasive lobular carcinoma, medullary carcinoma, colloid carcinoma (mucinous carcinoma), Paget's disease, tubular carcinoma, adenoid cystic carcinoma, invasive comedocarcinoma, apocrine carcinoma, and invasive papillary carcinoma. See, also, Cancer, Principles and Practice of Oncology, DeVita et al., ed., J.B. Lippincott Company, 1982, Pages 914-922. The different cancers can generally be distinguished histologically from each other.

Over 90% of breast cancers arise in the ducts. As long as it remains with the ductal basement membranes, it is classified as a non-infiltrating or non-invasive carcinoma. DCIS is a common example. An invasive or infiltrating carcinoma shows a marked increase in dense fibrous tissue stroma, giving the tissue a hard consistency. IDC is one of the more common types of an invasive carcinoma. Frequently, an infiltrating carcinoma becomes invaded with blood and lymphatic vessels as it increases in size and malignancy. The tumor cells fill the ducts, plugging them, and invade the surrounding stroma. For general description of breast pathology, see, e.g., Robins Pathological Basis of Disease, Cotran et al., 4^{th} Edition, W.B. Saunders Company, 1989, Chapter 25.

The progression of a cancer, from its origin to a full-blown malignancy, is the subject of intense study. Hyperplasia is generally believed to precede at least some cancers, but not all hyperplasia leads to cancer, and the relationship between the two is not well understood. One hallmark of a hyperplasia that leads to cancer may be the occurrence of genomic instability, and other factors which lead to uncoupling of the cell cycle.

Intraepithelial neoplasia is one of the first detectable signs of a breast cancer, characterized by its confinement to the duct epithelia. It can also be referred to as preinvasive neoplasia, precancer, dysplasia, or CIS. See, e.g., Boone et al., Proc. Soc. Exp. Biol. Med., 216:151-165, 1997. An intraepithelial neoplasia generally consists of multiple foci of an abnormal clonal expansion of neoplastic cells. The development of the neoplasia is manifested by an increasing size of the lesion and a greater degree of cytonuclear morphological aberration, as it progresses from low grade to high grade. See, e.g., Bacus et al., Cancer Epid. Biom. Prevent., 8:1087-1094, 1999. An early grade can be referred to as an intraductal proliferation (IDP). More advanced, pre-invasive lesions are DCIS and LCIS (lobular carcinoma in situ). It is believed that DCIS and LCIS are precursor lesions of invasive breast cancer, such as IDC. See, e.g., Buerger et al., Mol. Pathol., 53:118-121, 2000.

Breast cancers can be both staged and graded. Stage is based on the tumor and size and whether the lymph nodes are involved with the tumor. Tumor grade refers to the tumor cells' appearance under the microscope, and how closely it resembles normal tissue of the same type. If the tumor cells look normal, then it can be termed “low grade.” High grade cells look markedly different from normal cells. High grade tumors tend to behave more aggressively than lower grade. An “ungraded” cancer indicates that the gene expression profile as described herein indicates that it has an expression profile of group DI genes.

The most widely used clinical staging system for breast cancer is one adopted by the UICC (International Union against Cancer). This system incorporates the TNM (t, tumor; N, nodes; M, metastases) classification using tumor size, involvement of the chest wall and skin, inflammatory cancer, involvement of nodes, evidence of metastases. See, e.g., Sainsbury et al., BMJ, 321:745-750, 2000. Other staging and grading systems can also be used, e.g., Bloom and Richardson grade (British J. Cancer, 11:359-377, 1957), Columbia Clinical Classification (CCC), Van Nuys (VN), etc. Grading systems have also been devised based on image analysis of neoplastic and normal cells. Bacus et al. (Cancer Epid. Biom. Prevent., 8:1087-1094, 1999) have described an image morphometric nuclear grading system for intraepitheliam neoplastic lesions, such as DCIS, which provides objective criteria to assess tumor grade. See, also, Schwartz, Human Pathol., 28:1798-1802, 1997, for a grading system for DCIS. FISH has also been used to diagnose cancers based on chromosomal aberrations. See, e.g., Komoike et al., Breast Cancer, 7:332-336, 2000.

Various genetic bases for breast cancer have begun to be identified. For instance, BRCA1, BRCA2, ATM, PTEN/MMAC1 (e.g., Ali et al., J. Natl. Cancer Inst., 91:1922-1932, 1999), MLH2, MSH2, TP53 (e.g., Done et al., Cancer Res., 58:785-789, 1998), and STK11 are associated with a higher risk of cancer. Other genes involved in breast cancer include, e.g., myc, cyclin D1 (e.g., Weinstat-Saslow et al., Nature Med., 1:1257-1260, 1995), and c-erb-B2.

Grading, Staging, Comparing, Assessing, Methods and Compositions

The present invention also relates to methods and compositions for staging and grading cancers. As already defined, staging relates to determining the extent of a cancer's spread, including its size and the degree to which other tissues, such as lymph nodes are involved in the cancer. Grading refers to the degree of a cell's retention of the characteristics of the tissue of its origin. A lower grade cancer comprises tumor cells that more closely resemble normal cells than a medium or higher grade cancer. Grading can be a useful diagnostic and prognostic tool. Higher grade cancers usually behave more aggressively than lower grade cancers. Thus, knowledge of the cancer grade, as well as its stage, can be a significant factor in the choice of the appropriate therapeutic intervention for the particular patient, e.g., surgery, radiation, chemotherapy, etc. Staging and grading can also be used in conjunction with a therapy to assess its efficacy, to determine prognosis, to determine effective dosages, etc.

Various methods of staging and grading cancers can be employed in accordance with the present invention. Table 1 provides examples of the cell expression profiles of two graded cancers (D for DCIS and I for IDC) for about 269 genes. A “cell expression profile” or “cell expression fingerprint” is a representation of the expression levels of various different genes in a given cell or sample comprising cells. DCIS represents a lower grade breast cancer and IDC represent a higher grade breast cancer. The cell expression profiles of DCIS and IDC in Table 1 reflect only those genes that have been determined to be up-regulated in comparison to a sample from normal breast tissue. DCIS has a cell expression profile that comprises, for instance, lower up-regulated expression of BCU36, BCU38, and BCU99, medium up-regulated expression of BCU135, BCU579, and BCU893, and higher up-regulated expression of BCU470. These cell expression profiles can be useful as reference standards. For instance, the cell expression profiles of samples (e.g., a biopsy sample obtained from a patient, cancer cells circulating in the blood or lymph) can be compared to the DCIS and IDC profiles as standards for lower and higher grade cancers, respectively, to determine which grade the sample most closely resembles. The cell expression fingerprints can be used alone for grading, or in combination with other grading methods.

A cell expression profile can consist of the expression pattern of a breast tissue sample for differentially-regulated genes selected from: group D genes, SEQ NOS 1-3 and 188-225, for DCIS or a low grade cancer; group I genes, SEQ 226-269, for IDC or a high grade cancer, and group DI genes, SEQ NOS 4-187, for an ungraded cancer. The phrase “expression pattern” or “expression profile” as used throughout indicates the picture of those genes whose expression can be detected, e.g., as here, in the sample tissue.

The profiles in Table 1, and other profiles of the genes represented by SEQ NOS 1-269 can be used in a method for grading a breast cancer in a sample comprising cells, comprising one or more of the following steps in any effective order, e.g., determining the expression levels of target genes in said sample, wherein said target genes comprise genes represented by sequences selected from SEQ NOS 1-269, and comparing said expression levels of said target genes to the cell expression profile of a lower grade cancer or a higher grade cancer, wherein said profiles are shown in Table 1.

For any of the uses mentioned herein this disclosure, the genes can be analyzed, assessed, detected, etc., by any combinations, groups, sets, subsets, etc., e.g., all D's, DL, DM, DH, all I's, IL, IM, IH, all DI's, DIL, DIM, DIH, functional groups, such as transcription factors, cell-cycle regulatory proteins, proteases, adhesion proteins, cytokines and cytokine receptors, cell-surface proteins, membrane channels and transporters, enzymes, etc.

Expression levels refer to the amounts of RNA or polypeptide produced by transcription and translation, respectively. The phrase “expression level” as used in this disclosure refers to an amount or quantity (e.g., high, low, medium, etc.) of a product of the gene of interest (mRNA, polypeptide, etc.) which appears in the cell or tissue when the gene is active. These amounts can be determined in accordance with any suitable method, including those already mentioned, such as Northern blot analysis, polymerase chain reaction (PCR), reverse transcriptase PCR, RACE PCR, or in situ hybridization. The levels can be determined in the same or different method. For instance, expression levels of target genes can be determined independently, e.g., using a gene chip array or by a contracting laboratory, etc., and then those results can be used in a grading method.

Once obtained, the expression levels of the genes can be compared to a cell expression profile of a reference standard, such as a graded cancer or a normal tissue. The comparison can be conducted for different purposes, e.g., as a control to determine reproducibility of the detection method, to establish whether the sample comprises cells of the same origin as the reference standard, etc. For grading the cells contained in the sample, the expression levels can be compared to one or more cell expression profiles of graded cancers, such as a lower grade cancer and/or a higher grade cancer as shown in Table 1. Comparing can be accomplished using all genes, or only certain gene subsets, e.g., genes which are uniquely expressed in a low or high grade cancer (i.e., omitting some or all genes expressed in both cancer grades), etc.

A method of the present invention for grading a breast cancer in a sample comprising cells can also comprise one or more the following steps in any effective order, e.g., determining the expression levels of target genes in said sample, wherein said target genes comprise genes represented by sequences selected from SEQ NOS 1-269; assessing whether the expression levels most closely match the cell expression profile of a lower grade breast cancer or a higher grade breast.

Grading can be accomplished by assessing whether the expression levels in the sample most closely match the cell expression profile of a graded cancer, such as a lower grade breast cancer and/or a higher grade breast. By the phrase “assessing,” it is meant, e.g., comparing, analyzing, evaluating, etc. In other words, the expression levels of a sample are compared to one or more standards to determine whether and what standard it “most closely matches.” If it matches a lower grade standard more closely than a higher grade standard, then the sample is assessed as being a lower grade cancer. The method is not to be limited to how the assessing is accomplished.

The phrase “most closely matches,” indicates, e.g., that the profile or fingerprint of the sample may not be identical to the standard (e.g., DCIS or IDC), but resembles one cell expression pattern over another. The methods are not limited to how the degree of match is determined. Various algorithms can be used to assess pattern similarities between a sample and a standard. See, e.g., U.S. Pat. 4,981,783.

In the example shown in Table 3, expression levels of ten target genes were determined. The expression profiles of these genes are listed in Table 1. A plus (+) indicates that the gene was up-regulated in the sample. A blank indicates that the gene was not up-regulated in the sample when compared to the same gene in a normal tissue. BCU135 and BCU470 are up-regulated in the lower grade cancer and the sample. BCU540 and BCU926 are not up-regulated in the lower grade cancer nor in the sample. Thus, the sample behaves like the lower grade cancer for 4 of the genes. On the other hand, BCU886 is up-regulated in both the higher grade cancer and the sample. BCU442 and BCU227 are not up-regulated (e.g., not expressed or expressed at same levels) in both; the higher grade cancer and the sample. The sample behaves like a higher grade cancer for 3 of the genes. The sample gene expression fingerprint is assessed as more closely resembling or matching the lower grade tumor because it contains a greater number of genes that behave like the lower grade cancer than the higher grade cancer. The sample can also be characterized as a transitional stage between a lower and high grade cancer because there are both types of genes up-regulated in the sample. This could be useful prognostically. For instance, if an earlier biopsy had shown that the profile was predominantly higher grade genes, the switch to lower grade cancer genes could suggest treatment efficacy.

In addition, the present invention relates to methods of assessing a therapeutic or preventative intervention in a subject having a cancer, comprising, e.g., detecting the expression levels of up-regulated target genes, wherein the target genes comprise a gene which is represented by a sequence selected from SEQ NOS 1-269, or, a gene represented by a sequence having 95% sequence identity or more to a sequence selected from SEQ NOS 1-269. By “therapeutic or preventative intervention,” it is meant, e.g., a drug administered a patient, surgery, radiation, chemotherapy, and other measures taken to prevent a cancer or treat a cancer.

Arrays

The present invention also relates to an ordered array of polynucleotide probes, polypeptides, or specific-binding partners thereto for detecting the expression of differentially expressed breast cancer genes in a sample, comprising, polynucleotide, polypeptide, or specific-binding partner probes associated with a solid support, wherein each probe is specific for a different differentially expressed breast cancer gene, and the probes comprise a nucleotide sequence of SEQ NOS 1-269 which is specific for said gene, a nucleotide sequence having sequence identity to SEQ NOS 1-269 which is specific for said gene or polynucleotide, or complements thereto, or polypeptides encoded thereby, or specific-binding partners thereto. Ordered arrays can comprise subsets of genes, polypeptides, specific-binding partners, e.g., genes up-regulated in DCIS, genes up-regulated in IDC, genes up-regulated in both DCIS and IDC, all D's, DL, DM, DH, all I's, IL, IM, IH, all DI's, DIL, DIM, DIH, functional groups, such as transcription factors, cell-cycle regulatory proteins, proteases, adhesion proteins, cytokines and cytokine receptors, cell-surface proteins, membrane channels and transporters, enzymes, etc., genes listed in Table 2, etc.

The phrase “ordered array” indicates that the probes, polypeptides, specific binding partners, etc., are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes, etc., are associated with the solid support in any effective way. For instance, the probes, etc., can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes, etc., can fill the hollow orifice, be attached to the surface of the orifice, etc. Probes, etc., can be of any effective size, sequence identity, composition, etc., as already discussed.

Polynucleotide Expression, Polypeptides Produced Thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, 293, ZR-75-1 (ATCC CRL-1500), ZR-75-30 (ATCC CRL-150), UACC-812 (ATCC CRL-1897), UACC-893 (ATCC CRL-1902), HCC38 (ATCC CRL-2314), HCC70 (CRL-2315), and other HCC cell lines (e.g., as deposited with the ATCC), AU565 (ATCC CRL-2351), Hs 496.T (ATCC CRL-7303), Hs 748.T (ATCC CRL-7486), SW527 (ATCC CRL-7940), 184A1 (ATCC CRL-8798), MCF cell lines (e.g., 10A and others deposited with the ATCC), MDA-MB-134-VI (ATCC HTB-23 and other MDA cell lines), SK-BR-3 (ATCC HTB-30), ME-180 (ATCC HTB-33), Hs 578Bst (ATCC HTB-125), Hs 578T (ATCC HTB-126), T-47D (ATCC HTB-133), insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in SEQ NOS 1-269, or it can contain degenerate codons coding for the same amino acid sequences. For instance, it may be desirable to change the codons in the sequence to optimize the sequence for expression in a desired host.

Antisense

Antisense polynucleotide (e.g., RNA) can also be prepared from a polynucleotide according to the present invention, preferably an anti-sense to a sequence of SEQ NOS 1-269. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,153,595, 6,133,246, 6,117,847, 6,096,722, 6,087,343, 6,040,296, 6,005,095, 5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); U.S. Pat. No. 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′-O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Identifying Agent Methods

The present invention also relates to methods of identifying agents, and the agents themselves, which modulate SEQ NOS 1-269. These agents can be used to modulate the biological activity of the polypeptide encoded for the gene, or the gene, itself. Agents which regulate the gene or its product are useful in variety of different environments, including as medicinal agents to treat or prevent disorders associated with SEQ NOS 1-269 and as research reagents to modify the function of tissues and cell.

Methods of identifying agents generally comprise steps in which an agent is placed in contact with the gene, transcription product, translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of a gene selected from SEQ NOS 1-269, a method can comprise, in any effective order, one or more of the following steps, e.g., contacting a SEQ NOS 1-269 gene (e.g., in a cell population) with a test agent under conditions effective for said test agent to modulate the expression of SEQ NOS 1-269, and determining whether said test agent modulates said SEQ NOS 1-269. An agent can modulate expression of SEQ NOS 1-269 at any level, including transcription, translation, and/or perdurance of the nucleic acid (e.g., degradation, stability, etc.) in the cell.

For modulating the biological activity of SEQ NOS 1-269 polypeptides, a method can comprise, in any effective order, one or more of the following steps, e.g., contacting a SEQ NOS 1-269 polypeptide (e.g., in a cell, lysate, or isolated) with a test agent under conditions effective for said test agent to modulate the biological activity of said polypeptide, and determining whether said test agent modulates said biological activity.

Contacting SEQ NOS 1-269 with the test agent can be accomplished by any suitable method and/or means that places the agent in a position to functionally control expression or biological activity of SEQ NOS 1-269 present in the sample. Functional control indicates that the agent can exert its physiological effect on SEQ NOS 1-269 through whatever mechanism it works. The choice of the method and/or means can depend upon the nature of the agent and the condition and type of environment in which the SEQ NOS 1-269 is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

After the agent has been administered in such a way that it can gain access to SEQ NOS 1-269, it can be determined whether the test agent modulates SEQ NOS 1-269 expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate SEQ NOS 1-269 expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc. Biological activities of SEQ NOS 1-269 include, e.g., ligand binding, etc.

A test agent can be of any molecular composition, e.g., chemical compounds, biomolecules, such as polypeptides, lipids, nucleic acids (e.g., antisense to a polynucleotide sequence selected from a gene of SEQ ID NOS 1-269), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity a polypeptide in a lysate or other cell-free form. Antisense SEQ NOS 1-269 can also be used as test agents to modulate gene expression.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving differentially expressed breast cancer gene sequences from a computer-readable medium, comprising, one or more of the following steps in any effective order, e.g., selecting a cell or gene expression profile, e.g., a profile that specifies that said gene is differentially expressed in breast, and retrieving said differentially expressed breast cancer gene sequences, where the gene sequences consist of the genes represented by SEQ NOS 1-269, or, e.g., all D's, DL, DM, DH, all I's, IL, IM, IH, all DI's, DIL, DIM, DIH.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in breast. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in a lower grade cancer. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Markers

The polynucleotides of the present invention can be used with other markers, especially breast and breast cancer markers to identity, detect, stage, diagnosis, determine, prognosticate, treat, etc., tissue, diseases and conditions, etc, of the breast. Markers can be polynucleotides, polypeptides, antibodies, ligands, specific binding partners, etc. The targets for such markers include, but are not limited genes and polypeptides that are selective for cell types present in the breast. Specific targets include, BRCA1, BRCA2, ATM, PTEN/MMAC1 (e.g., Ali et al., J. Natl. Cancer Inst., 91:1922-1932, 1999), MLH2, MSH2, TP53 (e.g., Done et al., Cancer Res., 58:785-789, 1998), STK11, myc, cyclin D1 (e.g., Weinstat-Saslow et al., Nature Med., 1:1257-1260, 1995), c-erb-B2, keratins, such as 5/6 and 8/18.

Therapeutics

Selective polynucleotides, polypeptides, and specific-binding partners thereto, can be utilized in therapeutic applications, especially to treat diseases and conditions of the breast. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a breast-specific antigen on the cell-surface can be used to stimulate the body to destroy or attack the cancer, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partners can be conjugated to a second molecule, such as a cytotoxic agent, and used for targeting the second molecule to a breast-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well known (see, e.g., Arnon et al.; Reisfeld et al., 1985; Hellstrom et al.; Robinson et al., 1987; Thorpe, 1985; and Thorpe et al., 1982).

In addition to immunotherapy, polynucleotides and polypeptides can be used as targets for non-immunotherapeutic applications, e.g., using compounds which interfere with function, expression (e.g., antisense as a therapeutic agent), assembly, etc.

Delivery of therapeutic agents can be achieved according to any effective method, including, liposomes, viruses, plasmid vectors, bacterial delivery systems, orally, systemically, etc.

Antibodies to cell-surface antigens can also be used in imaging breast tissue. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos, 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a binding partner with an agent for any desired purpose.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

Although this disclosure is written in terms of breast cancer, it is not to be limited to breast cancer. Cancers derived from other tissue types can differentially express any of the disclosed sequences and genes, making the methods (diagnosis, staging, grading, treatment, therapeutic, etc.) generally applicable to the cancer field.

The topic headings set forth above are meant as guidance where certain information can be found in the application, but are not intended to be the only source in the application where information on such topic can be found.

Reference Materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the invention to its fullest extent. The entire disclosure of all applications, patents and publications, cited above and in the figures are hereby incorporated by reference in their entirety.

1. A method for diagnosing a breast cancer in a sample comprising tissue, comprising:
determining the number of target genes which are up-regulated in said sample, wherein said target genes are selected from SEQ NOS 1-269 of claim 22,
whereby said number is indicative of the probability that said sample comprises breast cancer.

2. A method of claim 1, wherein said determining is performed by Northern blot analysis, polymerase chain reaction (PCR), reverse transcriptase PCR, RACE PCR, or in situ hybridization using polynucleotide probes specific for polynucleotide sequences selected from SEQ NOS 1-269.

3. A method of claim 1, wherein said determining is performed by:
contacting said sample with a polynucleotide probe under conditions effective for said probe to hybridize specifically to a target nucleic acid in said sample,
detecting the amount of hybridization between said probe and target nucleic acid, and
comparing the amount of hybridization in said sample with the amount of hybridization of said probe in a second sample comprising normal breast tissue.

4. A method of claim 1, wherein said determining is performed by:
contacting said sample with a polynucleotide probe under conditions effective for said probe to hybridize specifically to a target nucleic acid in said sample,
detecting the amount of hybridization between said probe and target nucleic acid, and
comparing the amount of hybridization in said sample with the amount of hybridization between a second probe and its corresponding second target nucleic acid in said sample.

5. A method of claim 2, wherein said probe is a contiguous sequence of at least 8 nucleotides selected from a polynucleotide sequence selected from SEQ NOS 1-269 of claim 22, or a complement thereto.

6. A method of assessing a therapeutic or preventative intervention in a subject having breast cancer, comprising:
determining the expression levels in a sample comprising breast tissue of target genes which are differentially-regulated in breast cancer,
wherein said target genes are selected from SEQ NOS 1-269 of claim 22.

7. A method of claim 6, wherein the expression levels of at least 10 genes are determined.

8. A method of claim 6, wherein the determining is performed by Northern blot analysis, polymerase chain reaction (PCR), reverse transcriptase PCR, RACE PCR, or in situ hybridization using polynucleotide probes specific for polynucleotide sequences selected from SEQ NOS 1-269.

9. A method of claim 6, wherein said determining is performed by:
contacting said sample with a polynucleotide probe under conditions effective for said probe to hybridize specifically to a target nucleic acid in said sample,
detecting the amount of hybridization between said probe and target nucleic acid, and
comparing the amount of hybridization in said sample with the amount of hybridization of said probe in a second sample comprising normal breast tissue.

10. A method of identifying agents that modulate the expression of polynucleotides up-regulated in breast cancer cells, comprising,
contacting a cell population with a test agent under conditions effective for said test agent to modulate the expression of a polynucleotide in said cell population, and
determining whether said test agent modulates said polynucleotide expression, wherein said polynucleotide is selected from SEQ NOS 1-269 of claim 22.

11. A method of claim 10, wherein said agent is a polynucleotide which is antisense and effective to inhibit translation of the polynucleotide.

12. A method for grading a breast cancer in a sample comprising cells, comprising:
determining the expression levels of target genes in said sample, wherein said target genes are selected from SEQ NOS 1-269 of claim 22, and
assessing whether the expression levels most closely match the cell expression profiles of a low grade breast cancer, high grade breast cancer, or ungraded breast cancer, whereby said cancer is graded,
wherein group D genes, SEQ NOS 1-3 and 188-225, are for a low grade cancer, group I genes, SEQ 226-269, are for a high grade cancer, and group DI genes, SEQ NOS 4-187, are for a ungraded cancer.

13. A methods of claim 12, wherein said determining is performed by Northern blot analysis, polymerase chain reaction (PCR), reverse transcriptase PCR, RACE PCR, or in situ hybridization using polynucleotide probes specific for polynucleotides sequences selected from SEQ NOS 1-269.

14. A method of claim 13, wherein said determining is performed by:
contacting said sample with a polynucleotide probe under conditions effective for said probe to hybridize specifically to a target nucleic acid in said sample,
detecting the amount of hybridization between said probe and target nucleic acid, and
comparing the amount of hybridization in said sample with the amount of hybridization of said probe in a second sample comprising normal breast tissue.

15. A method of claim 13, wherein said determining is performed by:
contacting said sample with a polynucleotide probe under conditions effective for said probe to hybridize specifically to a target nucleic acid in said sample,
detecting the amount of hybridization between said probe and target nucleic acid, and
comparing the amount of hybridization in said sample with the amount of hybridization between a second probe and its corresponding second target nucleic acid in said sample.

16. A method of claim 13, wherein said probe is a contiguous sequence of at least 8 nucleotides.

17. An ordered array of polynucleotide probes for detecting the expression of differentially regulated cancer breast genes in a sample, comprising:
polynucleotide probes associated with a solid support, wherein each probe is specific for a different up-regulated breast cancer gene, and the polynucleotide probes are specific for polynucleotides sequences selected from SEQ NOS 1-269.

18. An ordered array of claim 17, wherein each probe is a contiguous sequence of at least 8 nucleotides.

19. An ordered array of claim 17, comprising probes for low grade cancer, high grade cancer, and ungraded cancer.

20. A cell expression profile consisting of the expression pattern of a breast tissue sample for differentially-regulated genes of claim 22.

21. A cell expression profile of claim 20, comprising the expression levels of genes for each of a low grade, high grade, and ungraded cancer.

22. One or more polynucleotides which are differentially regulated in a breast cancer, selected from:
group D genes, SEQ NOS 1-3 and 188-225, for DCIS or a low grade cancer,
group I genes, SEQ 226-269, for IDC or a high grade cancer, and
group DI genes, SEQ NOS 4-187, for an ungraded cancer.

23. Polynucleotides of claim 22, selected from each of groups a)-c).
